(19) United States
(12) Patent Application Publication
(10) Pub. No.: US 2026/0165829 A1
(43) Pub. Date: Jun. 18, 2026

(54) Intraocular Artificial Lens Capsule

(71) Applicant: The Board of Trustees of the Leland Stanford Junior University, Stanford, CA (US)

(72) Inventors: Frank L. Brodie, Durham, NC (US); David Myung, Saratoga, CA (US); David Buickians, Glendale, CA (US)

(21) Appl. No.: 19/376,509

(22) Filed: Oct. 31, 2025

Related U.S. Application Data
(63) Continuation of application No. 17/284,578, now Pat. No. 12,458,487.
(60) Provisional application No. 62/806,772, filed on Feb. 16, 2019.

(51) Int. Cl. A61F 2/16; A61L 27/16

(52) U.S. Cl. CPC A61F 2/16; A61L 27/16; A61F 2002/1683; A61F 2002/169; A61F 2002/16901; A61F 2002/16902; A61F 2230/0041; A61F 2230/0052; A61F 2250/0067; A61F 2250/0091; A61L 2430/16

(57) ABSTRACT

An implantable artificial capsule and a method of implantation are provided. The implantable artificial capsule has a ring with a central opening to accommodate an intraocular lens and at least three haptic arms extending substantially orthogonal from the outer surface of the ring. Each arm has transcleral anchors for sutureless scleral fixation. Upon implantation of the implantable artificial capsule the arms are externalized trans-sclerally and atraumatically. The arms sit subconjunctivally. The implantable artificial capsule does not have any sutures for fixation and is suturelessly implanted in an eye, which is a major advantage as it significantly reduces complications during surgery and recovery.

### CROSS-REFERENCE TO RELATED APPLICATIONS

This application is a continuation of U.S. Patent Application Ser. No. 17/284,578 filed Apr. 12, 2021, now U.S. Pat. No. 12,458,487 issued on Nov. 4, 2025, which is incorporated herein by reference.

U.S. patent application Ser. No. 17/284,578 is a 371 of PCT application PCT/US2019/056020 filed Oct. 13, 2019.

PCT application PCT/US2019/056020 claims the benefit of U.S. Provisional application 62/749,262 filed Oct. 23, 2018.

PCT application PCT/US2019/056020 claims the benefit of .U.S Provisional application 62/806,772 filed Feb. 16, 2019.

### FIELD OF THE INVENTION

This invention relates to intraocular artificial lens capsules.

### BACKGROUND OF THE INVENTION

Implantation of an intraocular lens (IOL) requires support within the eye to hold it in the correct position. Normally, this is achieved through the native capsular bag suspended by the zonules (fine thread-like structures). However, these support structures can be compromised either due to intrinsic factors such as pseudoexfoliation, Marfan, or Weill Marchesani syndromes, or extrinsic factors such as trauma. Additionally, lens support can be compromised iatrogenically either during the time of surgery (either anterior or posterior segment surgery) or as a late complication of previous surgery.

The management of secondary IOL placement in the absence of sufficient capsular or zonular support continues to evolve. Currently, several options exist, each with significant drawbacks. The anterior chamber Intraocular Lens (ACIOL) is a larger lens with the ability to sit anterior to the iris, however, over time these lenses can cause uveitis, glaucoma, hyphema (UGH) syndrome as well as endothelial cell loss and corneal decompensation. Iris sutured lenses are technically difficult and can lead to iris pigment loss causing glaucoma. Lastly, eye wall (scleral) sutured lenses are technically complex, risk rotation, and the durability of the sutures is unknown, with reported cases of breakage. Additionally, all these techniques force the surgeon to use an alternative lens type not their preferred lens for the patient. Lastly, the decision of timing is critical—frequently lens calculations are inadequate during the initial vitrectomy/lensectomy yet there is the desire to not subject the patient to additional posterior segment surgery, so non-ideal lenses are frequently implanted. An ideal solution would replicate normal anatomy, be technically easy, provide long term secure placement and allow for lens selection with accurate preoperative calculations without requiring additional vitreoretinal surgery. The present invention provides a solution in that direction.

### SUMMARY OF THE INVENTION

The present invention provides an implantable artificial capsule. The implantable artificial capsule has a ring with a central opening to accommodate an intraocular lens and at least three haptic arms extending substantially orthogonal from the outer surface of the ring.

Each arm has transcleral anchors for sutureless scleral fixation. Upon implantation of the implantable artificial capsule the arms are externalized trans-sclerally and atraumatically. The arms sit subconjunctivally. The implantable artificial capsule does not have any sutures for fixation and is suturelessly implanted in an eye, which is a major advantage as it significantly reduces complications during surgery and recovery.

The transcleral anchors of each arm are T-shaped, anchor-shaped or fish-hook shaped, or nail-head-shaped. In one embodiment, the central opening has an inward facing groove to accommodate intraocular lens haptics or other intraocular implants.

In another aspect of the invention, a method of implanting an artificial capsule is provided. The method provides an artificial capsule as defined supra, implanting the artificial capsule within an eye, and suturelessly fixating each arm with their respective transcleral anchors. The arms upon implantation of the implantable artificial capsule are externalized trans-sclerally and atraumatically, and sit subconjunctivally of the eye. During surgery and after implantation, the implantable artificial capsule does not have any sutures for fixation.

Problems with prior art devices are, for example:
Anterior chamber intraocular lens: the lens is placed in front of the iris. These implants cause corneal decompensation, glaucoma and bleeding over time due to their instability in the eye.
Iris sutured lens: technically difficult and risks bleeding and glaucoma due to chafing of the iris.
Scleral sutured lens: technically difficult and cases of suture erosion/breakage require additional surgery and risk potentially blinding infection.

Embodiments of the invention have at least one or more of the following advantages:
Suture-less device which eliminates the risk of suture breakage.
A sutureless fixation method allowing for easier placement and secure attachment without concern for loosening or breaking of sutures.
A reliable refractive result based on known position without concern.
Posterior segment placement that greatly reduces risk of damage to iris, angle or cornea.
Ability to accommodate wide variety of intraocular lenses.
Replicates natural lens capsule.
Implantation posterior to iris and cornea eliminating or reducing risk of corneal injury, iris bleeding or glaucoma.
Device stably holds intraocular lens.
Because the device is a capsule only, the lens of choice can be implanted at the time of surgery or at a later date.
Haptics can be externalized as needed for scleral support/fixation.
Less complications than current technologies such as ACIOL, Iris-sutured lens, or Scleral-sutured lens.

### BRIEF DESCRIPTION OF THE DRAWINGS

FIG. 1 shows the artificial capsule according to exemplary embodiments of the invention.

FIGS. 2-5 show the artificial capsule and the physical implementation of the artificial capsule according to a first exemplary embodiment of the invention. The first exemplary embodiment pertains to an artificial capsule with a central opening that has an inward facing groove to accommodate intraocular lens haptics or other intraocular implants.

FIGS. 6-10 show the artificial capsule and the physical implementation of the artificial capsule according to a second exemplary embodiment of the invention. The second exemplary embodiment pertains to an artificial capsule that has a flat design with a central opening to accommodate intraocular lens haptics or other intraocular implants.

### DETAILED DESCRIPTION

The present invention is an artificial capsule with suture-less scleral fixation which would recapitulate normal anatomy. The artificial capsule has a central ring to support the intraocular lens while, in one embodiment, 3 haptics (arms) would be externalized trans-sclerally and sit subconjunctivally, ensuring a secure position while remaining covered to prevent erosion or infection. Sutureless placement would be rapid and technically straightforward. The artificial capsule would be made of a flexible biocompatible material such as silicon, polymethylmethacrylate or acrylic (both common IOL materials currently used). Other features of suitable materials are biocompatible, opaque or transparent. It could be inserted through a standard corneal incision and the haptics would be externalized through small gauge sclerotomy incisions. The capsule would accommodate a variety of common IOLs. The artificial capsule allows for the implantation of the IOL at the same time as placement, or IOL insertion could be delayed until a later date and only require a short anterior segment procedure for insertion.

### Applications

Embodiments of the invention have applications in cataract surgery when there is absent or severely compromised capsular support. This occurs in cases of trauma, surgical complication or with disease processes such as pseudoexfoliation or marfans syndrome.

The artificial capsule is intended to provide support and centration for an intraocular lens (IOL) without use of the native capsular bag. It is intended to be used in cases of deficient capsular and/or zonular support, either congenital or secondary to disease, trauma or iatrogenic injury. The artificial capsule can hold the lens in the sulcus plane or in the posterior chamber. The artificial capsule haptics can sit a-traumatically within the eye or one or more haptics can be externalized for scleral fixation. The IOL can be placed within the artificial capsule either at the time of implantation or at a later date. The IOL can be exchanged without removal of the artificial capsule.

### Prophetic Examples

A patient that sustained eye trauma leading to lens dislocation with complete loss of zonules undergoes lensectomy and vitrectomy. The artificial capsule is folded and inserted through a small standard corneal incision. The artificial capsule haptics are externalized through small gauge sclerotomies and are left subconjunctivaly to minimize irritation and infection risk. The patient recovers from surgery and once the eye is no longer inflamed undergoes appropriate biometry to determine correct intraocular lens implant. A simple subsequent procedure inserts the correct lens the artificial capsule.
A patient with pseudoexfoliation syndrome has a late IOL dislocation due to zonular instability. The patient undergoes vitrectomy and retrieval of the IOL. The artificial capsule is folded and inserted through a small standard corneal incision. The artificial capsule haptics are externalized through small gauge sclerotomies. The recovered IOL is placed in the artificial capsule.
A cataract surgery is complicated by 180 degrees of zonular loss. Sulcus lens placement is not possible and a history of glaucoma precludes Anterior Chamber IOL (ACIOL) placement. Scleral sutured lens would require an additional surgery by a vitreoretinal surgeon. Instead the artificial capsule is placed in the sulcus with 1 haptic externalized to provide support in the area of zonular loss. The IOL is then inserted into the artificial capsule.

### Design Examples

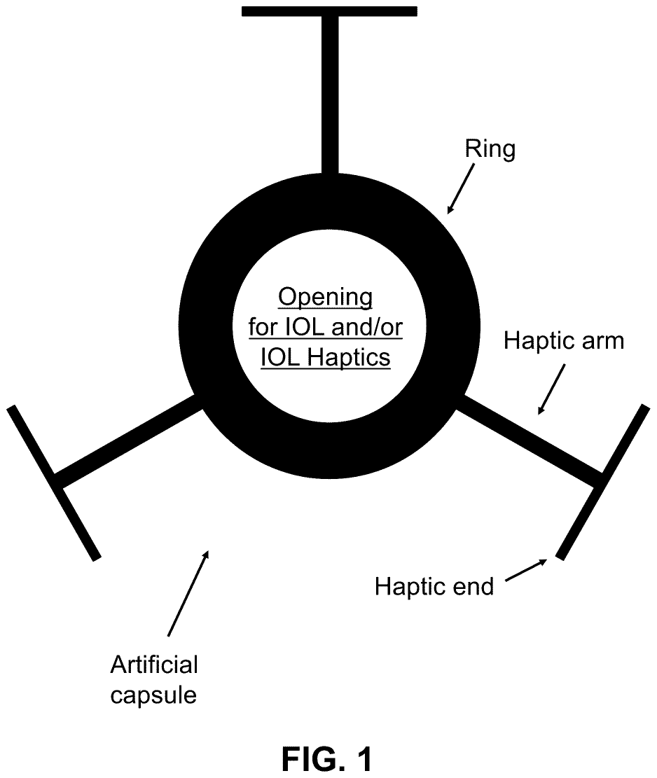

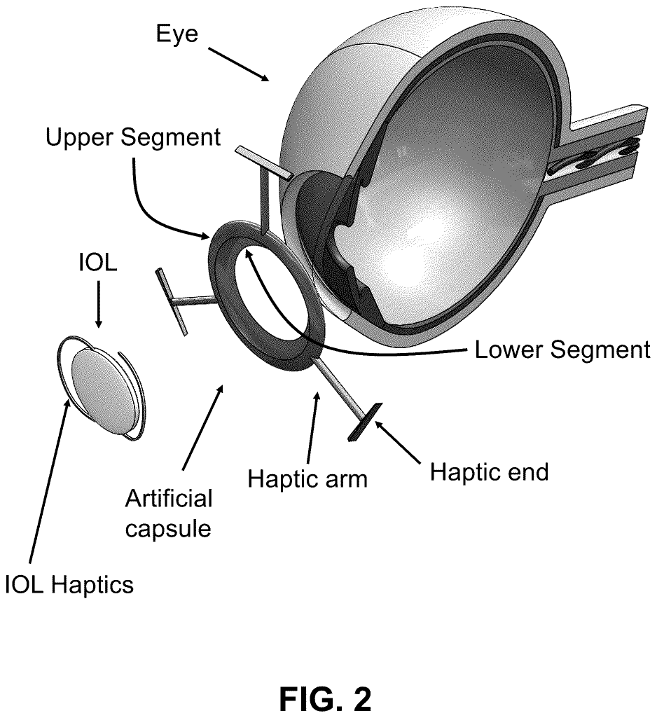

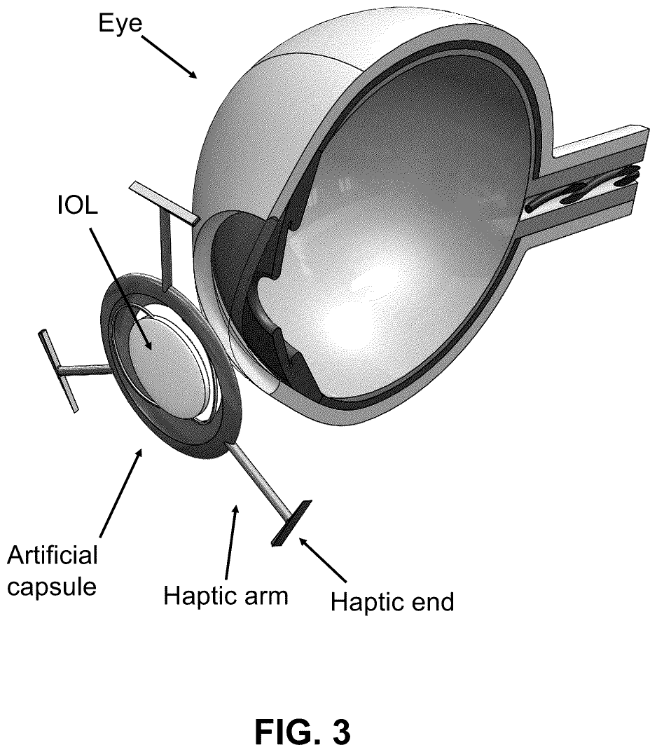

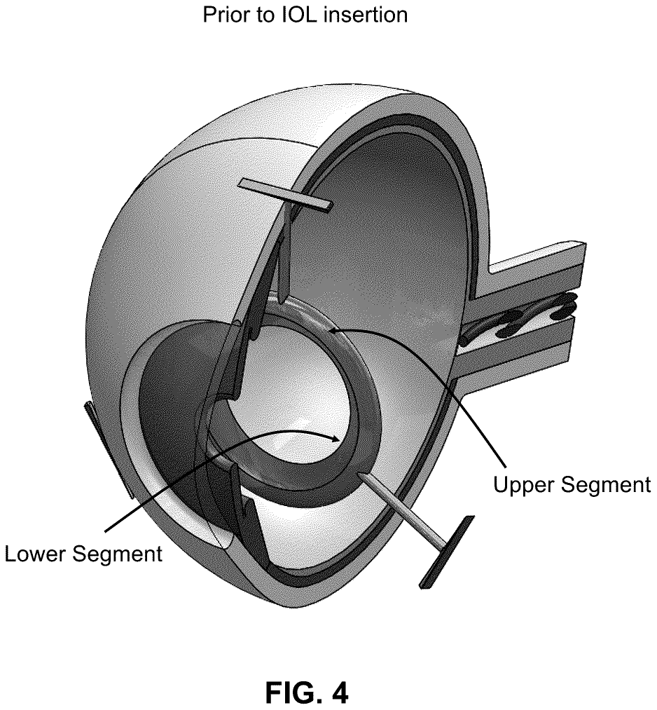

In a first example, the artificial capsule is a single object with two major components (FIGS. 1-4):
1. Circular ring with a central inward facing groove to accommodate the IOL and/or IOL haptics. The circular ring distinguishes:
   a. A lower segment—the flat base of the ring—diameter ranging from 5 mm to 10 mm with a central opening.
   b. An upper segment—overhang above the flat base—diameter ranging from 5 mm to 10 mm with a central opening.
   c. Both segments come together at the periphery in a point or rounded.
2. Haptics
   a. 3 or more haptics.
   b. each haptics is 0.75 mm to 0.01 mm thick.
   c. The end of each haptic is either T-shaped, anchor-shaped, fish-hook shaped or nail-head-shaped.

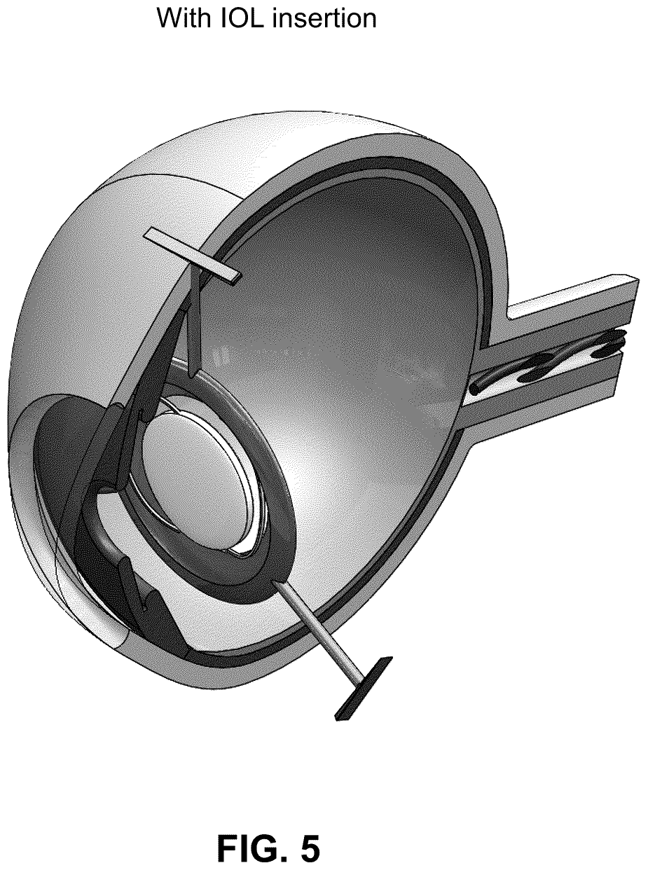

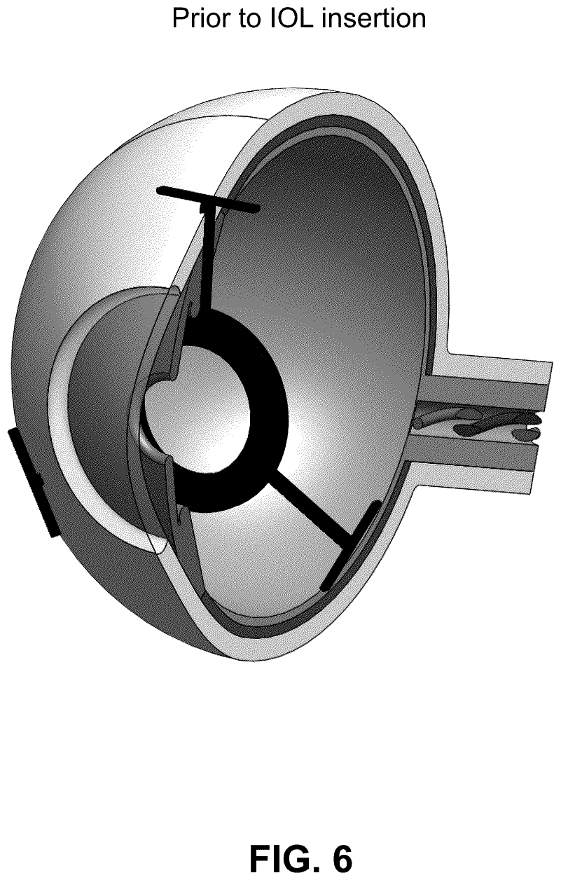

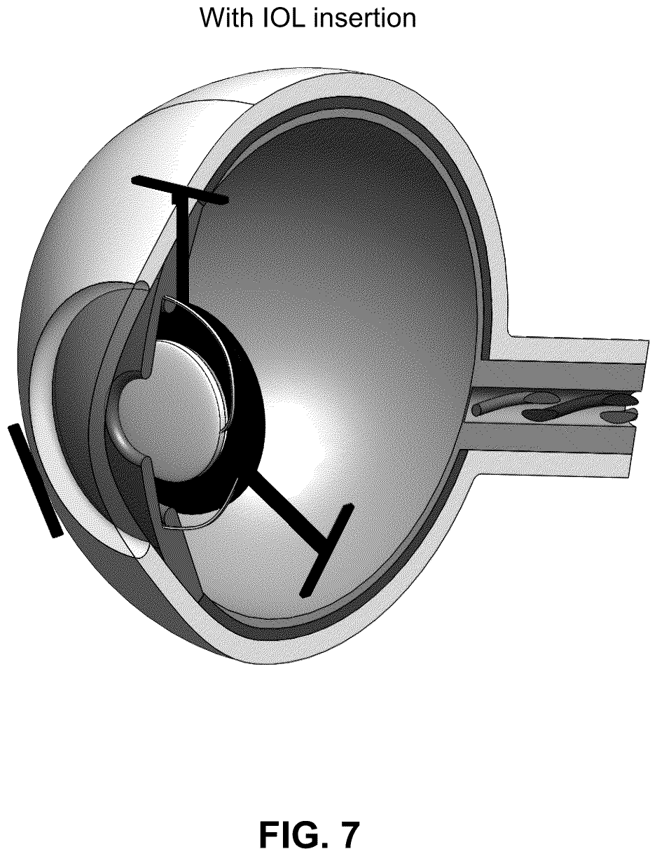

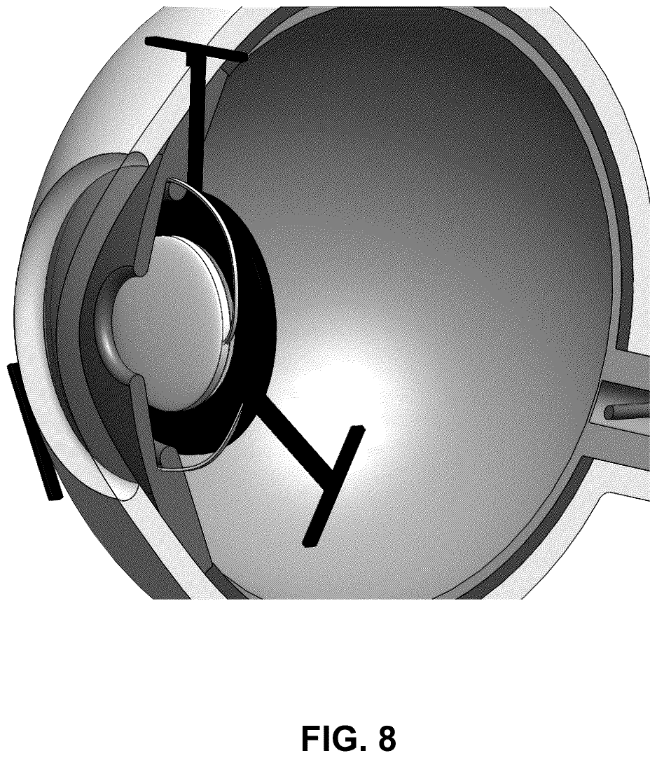

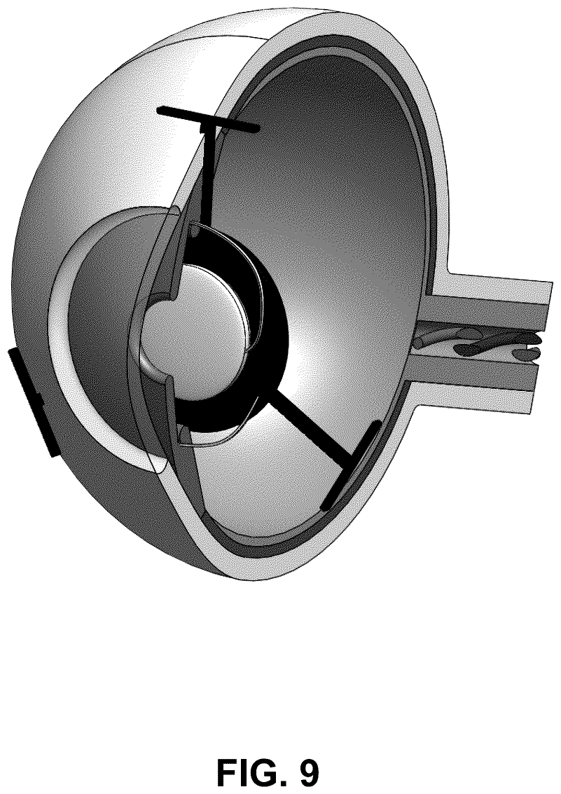

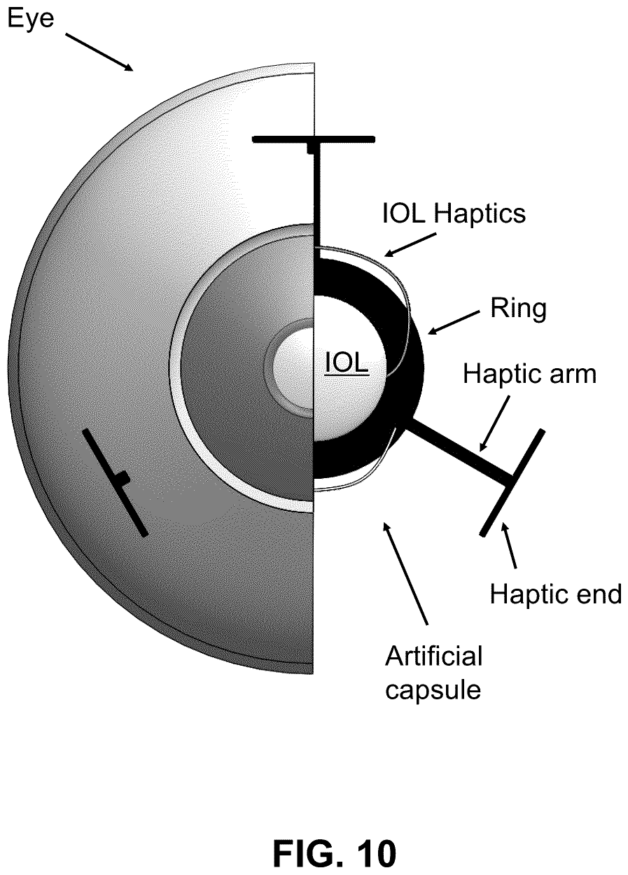

In a second example, the artificial capsule is a single object with two major components (FIGS. 5-10):
1. Flat circular ring where the flat ring part is designed to support IOL and/or IOL haptics.
   a. A single flat disk with a diameter ranging from 5 mm to 10 mm and a central opening.
3. Haptics
   a. 3 or more haptics.
   b. Each haptic is 0.75 mm to 0.01 mm thick.
   c. The end of haptic is either T-shaped, anchor-shaped, fish-hook shaped or nail-head-shaped.

Additional features to the first example could be: a second groove to support an additional implant. Additional features to either example could be an opaque colored overlying disc to serve as an artificial iris, drug eluting ring implant for long term posterior segment drug delivery, biosensor for continuous monitoring of intraocular characteristic (e.g. pressure, glucose, inflammation).

1. An implantable artificial capsule, comprising:
a structure comprising an outer, closed perimeter, a central opening, and a central, inward-facing groove for accommodating at least a portion of an intraocular lens or intraocular implant; and
at least three haptic arms, wherein prior to implantation, at least one haptic arm of said at least three haptic arms is straight and extends at a substantially orthogonal angle from the outer, closed perimeter of the structure,
wherein each haptic arm of said at least three haptic arms comprises only a single trans-scleral anchor for sutureless trans-scleral fixation upon implantation of the implantable artificial capsule in an eye, wherein each of said single trans-scleral anchors is located at a distal end of each of said at least three haptic arms to form a “T” shape.

2. The implantable artificial capsule as set forth in claim 1, wherein the structure further comprises a lower segment and an upper segment overhanging the lower segment, said lower segment joining said upper segment at a periphery to form said inward-facing groove of the structure, wherein said at least a portion of the intraocular lens accommodated by the inward-facing groove accommodates comprises one or more haptics of the intraocular lens.

3. The implantable artificial capsule as set forth in claim 2, wherein the periphery is rounded.

4. The implantable artificial capsule as set forth in claim 2, wherein the lower segment is planar.

5. The implantable artificial capsule as set forth in claim 4, wherein the lower segment supports a second portion of the intraocular lens.

6. The implantable artificial capsule as set forth in claim 2, wherein the lower segment and the upper segment each have an outer diameter that is about 5 mm to about 10 mm.

7. The implantable artificial capsule as set forth in claim 2, wherein the central opening of the structure is formed by a first opening through the lower segment and a second opening through the upper segment.

8. The implantable artificial capsule as set forth in claim 7, wherein the second opening through the upper segment is larger in diameter than the first opening through the lower segment.

9. The implantable artificial capsule as set forth in claim 1, wherein the structure comprises a flexible, biocompatible material.

10. The implantable artificial capsule as set forth in claim 9, wherein said flexible, biocompatible material comprises at least one of polymethylmethacrylate and acrylic.

11. The implantable artificial capsule as set forth in claim 9, wherein said flexible, biocompatible material comprises silicone.

12. The implantable artificial capsule as set forth in claim 1, wherein said structure is opaque or transparent.

13. The implantable artificial capsule as set forth in claim 1, wherein upon implantation into an eye, said capsule elutes a drug into said eye.

14. The implantable artificial capsule as set forth in claim 1, wherein the implantable artificial capsule comprises a first haptic arm, a second haptic arm, and a third haptic arm and the outer, closed perimeter of the structure has an outer surface.

15. The implantable artificial capsule as set forth in claim 14, wherein the first haptic arm begins at a first location along the outer surface of the outer, closed perimeter and ends at a second location opposite the first location of the outer surface, the first haptic arm extending at a substantially orthogonal angle between the first location and the second location,
wherein the second haptic arm begins at a third location along the outer surface of the outer, closed perimeter and ends at a fourth location opposite the third location of the outer surface, the second haptic arm extending at a substantially orthogonal angle between the third location and the fourth location, and
wherein the third haptic arm begins at a fifth location along the outer surface of the outer, closed perimeter and ends at a sixth location opposite the fifth location of the outer surface, the third haptic arm extending at a substantially orthogonal angle between the fifth location and the sixth location.

16. A method of implanting an artificial capsule, the method comprising:
positioning at least a portion of an artificial capsule within an eye, the artificial capsule comprising:
a structure having an outer, closed perimeter;
a central opening extending through the structure for accommodating at least a portion of an intraocular lens or intraocular implant; and
at least three haptic arms extending from the structure,
wherein each haptic arm of said at least three haptic arms comprises only a single trans-scleral anchor for sutureless trans-scleral fixation upon implantation of the artificial capsule in the eye, wherein each of the single trans-scleral anchors is located at a distal end of each of the at least three haptic arms to form a “T” shape, and
wherein prior to trans-scleral fixation, at least one haptic arm of the at least three haptic arms is straight and extends at a substantially orthogonal angle from the outer, closed perimeter of the structure; and
suturelessly externalizing the single trans-scleral anchors of each haptic arm of the at least three haptic arms.

17. The method as set forth in claim 16, wherein suturelessly externalizing the single trans-scleral anchors of each haptic arm comprises positioning the single trans-scleral anchors of each haptic arm to sit sub-conjunctivally.

18. The method as set forth in claim 16, further comprising positioning an implant within the artificial capsule.

19. The method as set forth in claim 18, wherein the implant comprises an intraocular lens or other intraocular implant.

20. The method as set forth in claim 18, wherein the structure further comprises a central, inward-facing groove for accommodating at least a portion of the implant within the artificial capsule.

21. The method as set forth in claim 18, wherein the implant is positioned within the artificial capsule during performing the method or after the method is performed.

22. The method as set forth in claim 18, wherein the implant is positioned within the artificial capsule after one haptic arm is fixated relative to the eye.

23. The method as set forth in claim 18, further comprising exchanging the implant for a second implant without removing the artificial capsule from the eye.

24. The method as set forth in claim 18, further comprising positioning a second implant within or on the artificial capsule.

25. The method as set forth in claim 24, wherein the second implant is an artificial iris, a drug-eluting ring implant, or a biosensor.

26. The method as set forth in claim 25, wherein the biosensor is for continuous monitoring of intraocular characteristic, the characteristic being at least one of pressure, glucose, and inflammation.

27. The method as set forth in claim 16, wherein positioning at least a portion of an artificial capsule within an eye comprises inserting the artificial capsule through a corneal incision.

28. The method as set forth in claim 27, wherein inserting the artificial capsule through the corneal incision comprises folding the artificial capsule and inserting through the corneal incision.

29. The method as set forth in claim 16, wherein suturelessly externalizing the single trans-scleral anchors of each haptic arm of the at least three haptic arms comprises externalizing through small gauge sclerotomy incisions.

30. The method as set forth in claim 16, wherein the eye has no native capsular bag, has deficient capsular and/or zonular support, either congenital or secondary to disease, trauma, or iatrogenic injury.
